(19) United States
(12) Patent Application Publication
(10) Pub. No.: US 2025/0025047 A1
(43) Pub. Date: Jan. 23, 2025

(54) Systems And Methods For Communicating With Patient In Magnetic Resonance Imaging Device

(71) Applicant: United States Government As Represented By The Department Of Veterans Affairs, Washington, DC (US)

(72) Inventors: Brandon B. Johnson, Salt Lake City, UT (US); Roger B. Kimball, Salt Lake City, UT (US)

(21) Appl. No.: 18/777,012

(22) Filed: Jul. 18, 2024

Related U.S. Application Data
(60) Provisional application No. 63/514,273, filed on Jul. 18, 2023.

(51) Int. Cl. A61B 5/00; A61B 5/055

(52) U.S. Cl. CPC A61B 5/0033; A61B 5/055; A61B 5/742; A61B 5/7455

(57) ABSTRACT

Apparatuses, systems, and methods for communicating a message to a patient in a magnetic resonance imaging (MRI) device are disclosed. An exemplary apparatus includes an indication source a conduit having a first end portion and a second end portion. The first end portion is in communication with the indication source. At least the second end portion is free of magnetic metal. The exemplary apparatus further includes an input device configured to receive an input from a medical professional. The input device is in operable communication with the indication source so that receipt of the input from the medical professional causes the indication source to output a communicating signal through the conduit.

### CROSS-REFERENCE TO RELATED APPLICATION

This application claims priority to and the benefit of the filing date of U.S. Provisional Patent Application No. 63/514,273, filed Jul. 18, 2023, the entirety of which is hereby incorporated by reference herein.

### FIELD

The present application relates to magnetic resonance imaging (MRI), and systems and methods for communicating with individuals (e.g., patients) positioned within MRI devices (e.g., scanners), particularly patients who are deaf or hard of hearing.

### BACKGROUND

Deaf and hard of hearing patients undergoing magnetic resonance imaging (MRI) face significant challenges due to the inability to follow breath hold instructions. This results in poor image quality, potential misdiagnosis, and the need for repeat exams. The absence of effective communication strategies and accommodations for these patients during the MRI process compromises the accuracy of the diagnostic outcomes, and also creates feelings of embarrassment and frustration among the patients themselves. This problem underscores the urgent need for innovative solutions and inclusive practices that address the needs of deaf and hard of hearing individuals. This will ensure equal access to high-quality diagnostic imaging services and improve overall healthcare experience.

It has been determined that no existing solution currently addresses the challenges faced by deaf and hard of hearing patients during MRI. Furthermore, it is evident that no viable solutions have been encountered thus far to address this specific problem. This scarcity of effective interventions necessitates the development of an innovative solution that caters to the needs of this patient population, ensuring improved imaging outcomes, accurate diagnoses, and an enhanced healthcare experience.

### SUMMARY

Disclosed herein, in various aspects, are apparatuses, systems, and methods for communicating a message to an individual (e.g., a patient) in a magnetic resonance imaging (MRI) device. An exemplary apparatus includes an indication source and a conduit having a first end portion and a second end portion. The first end portion is in communication with the indication source. At least the second end portion is free of magnetic metal. The exemplary apparatus further includes an input device configured to receive an input from a medical professional. The input device is in operable communication with the indication source so that receipt of the input from the medical professional causes the indication source to output a communicating signal through the conduit.

In exemplary aspects, a system comprises an MRI device and an apparatus including an indication source and a conduit having a first end portion and a second end portion. The first end portion is in communication with the indication source. At least the second end portion is free of magnetic metal. The apparatus further includes an input device configured to receive an input from a medical professional. The input device is in operable communication with the indication source so that receipt of the input from the medical professional causes the indication source to output a communicating signal through the conduit.

In another aspect, a method comprises the step of delivering, to an individual (e.g., a patent) in an MRI device, via a conduit having a second end portion that is free of magnetic metal and extends to an MRI device, a message in the form of one of pressurized air or light.

### BRIEF DESCRIPTION OF THE DRAWINGS

FIG. 1 is a schematic top plan view of a medical facility having an MRI device and an exemplary apparatus for communicating with an individual (a patient) in an MRI device.

FIG. 2 is a side view of an exemplary apparatus for communicating with an individual (a patient) in an MRI device as disclosed herein.

FIG. 3 is a system comprising an MRI device and the apparatus of FIG. 2.

FIG. 4 depicts exemplary instructions and messages (depicted in the form of a sticker) for attaching to a display device of the apparatus of FIG. 2.

FIG. 5 is a first array of symbols indicating, from left to right, breathing freely, preparing to hold breath, and holding breath.

FIG. 6 is a second array of symbols indicating, from left to right, breathing freely, preparing to hold breath, and holding breath.

FIG. 7A is an exemplary poor quality image of a patient who was breathing during imaging. FIG. 7B is an exemplary high quality image of a patient who was properly holding her breath during imaging.

FIG. 8 is a schematic view of an exemplary apparatus for communicating with an individual (a patient) in an MRI device as disclosed herein.

### DETAILED DESCRIPTION

The disclosed system and method may be understood more readily by reference to the following detailed description of particular embodiments and the examples included therein and to the Figures and their previous and following description.

It is to be understood that the terminology used herein is for the purpose of describing particular embodiments only and is not intended to limit the scope of the present invention which will be limited only by the appended claims.

It must be noted that as used herein and in the appended claims, the singular forms “a,” “an,” and “the” can optionally include plural references unless the context clearly dictates otherwise. Thus, for example, reference to “a screw” can represent disclosure of embodiments including only a single such screw, as well as embodiments including a plurality of such screws, and so forth.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Optionally, in some aspects, when values are approximated by use of the antecedents “about,” “substantially,” or “generally,” it is contemplated that values within up to 15%, up to 10%, up to 5%, or up to 1% (above or below) of the particularly stated value or characteristic can be included within the scope of those aspects.

Unless defined otherwise, all technical and scientific terms used herein have the same meanings as commonly understood by one of skill in the art to which the disclosed apparatus, system, and method belong. Although any apparatus, systems, and methods and materials similar or equivalent to those described herein can be used in the practice or testing of the present apparatus, system, and method, the particularly useful methods, devices, systems, and materials are as described.

MRI is important in diagnosing and characterizing life changing pathology. However, communication while performing an MRI can be necessary. Embodiments of the present disclosure can provide visual cues to effectively communicate messages to deaf and hard of hearing patients undergoing MRI scans. For example, MRI exams often involve breath-hold instructions and are important for accurate imaging. When patients are unable to follow these instructions, poor imaging can lead to misdiagnosis, misinterpretation, repeated exams, and non-diagnostic results, due to poor image quality. Currently, these instructions are solely provided audibly, which poses a challenge for these patients. By incorporating or tactile visual elements, the disclosed apparatuses and systems can bridge the communication gap and enhance patient comprehension during the procedure.

The disclosed apparatuses can be configured for use with existing MRI machines (e.g., existing MRI scanners). Accordingly, the disclosed apparatuses can be used without the need for modification to existing MRI machines. This capability permits widespread usage in a very fast and efficient manner to serve the patient population. The disclosed apparatuses can reduce repeat sequences, improve quality, minimize schedule delays, and improve patient experience. As further explained herein, the disclosed apparatuses, systems, and methods make use of current technology to create system optimizing Radiologist and Provider interaction with patients.

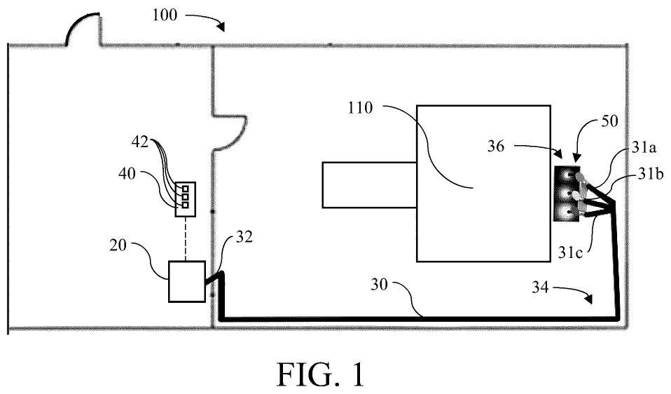

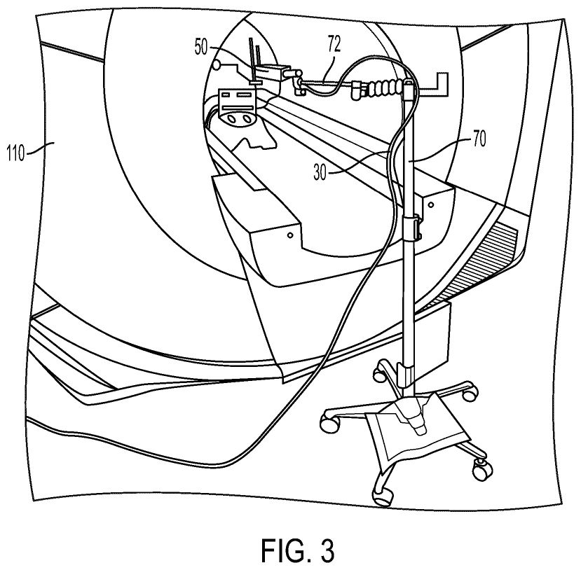

Referring to FIGS. 1-3, an exemplary apparatus 10 can be used with an MRI device 100 (e.g., an MRI scanner as is known in the art). The exemplary apparatus 10 can comprise an indication source 20. The exemplary apparatus 10 can further comprise a conduit 30 having a first end portion 32 and a second end portion 34. The first end portion can be in communication with the indication source 20. At least the second end portion 34 (or, optionally, an entirety of) the conduit 30 can be free of magnetic metal. In this way, the conduit 20 can extend to the MRI device without interfering with the MRI device during operation thereof. An input device 40 can be configured to receive an input from a medical professional. The input device 40 can be in operable communication with the indication source so that receipt of the input from the medical professional causes the indication source 20 to output a communicating signal through the conduit.

For example, in exemplary aspects, the indication source 20 can comprise at least one light source. In these aspects, the indication source 20 can comprise a light emitting diode (LED), a light engine, a laser, or a light strip (e.g., a plurality of LEDs), or combinations thereof. Accordingly, the conduit 30 can be a fiber optic cable, and the communicating signal can comprise light emitted from the light of the indication source 20. In exemplary, optional aspects, the fiber optic cable can be a multimode fiber optic cable.

In some optional aspects, the communicating signal can comprise a particular color of light. For example, a first color (e.g., green) can be indicative of a message to the individual (patient) in the MRI to breathe freely. A second color (e.g., red) can indicate a message to the individual (patient) in the MRI to hold his/her breath. Optionally, a third color (e.g., yellow) can indicate a message to the individual (patient) in the MRI to prepare to hold his/her breath soon. Accordingly, in some optional aspects, the at least one light can comprise a color changing light. For example, the color changing light can comprise a multicolor LED. In other optional aspects, the indication source 20 can comprise at least a first light and a second light. In other aspects, the fiber optic cable can be a first fiber optic cable 31a, and the first light can be configured to emit light through the first fiber optic cable. The apparatus 10 can further comprise a second fiber optic cable 31b, and the second light can be configured to emit light through the second fiber optic cable. In further aspects, the indication source 20 can comprise a third light, and the apparatus can comprise a third fiber optic cable 31c, wherein the third light is configured to emit light through the third fiber optic cable. The fiber optic cables can be bundled as a single multi-fiber cable. In other aspects, the fiber optic cables can be provided separately.

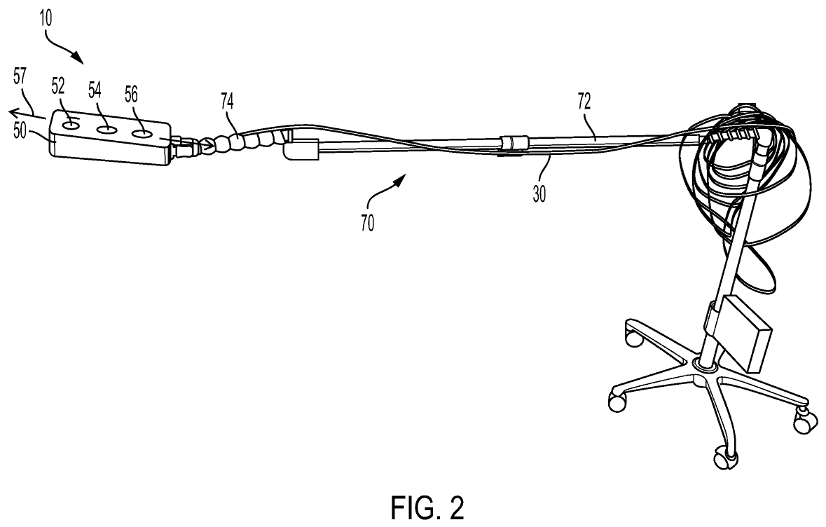

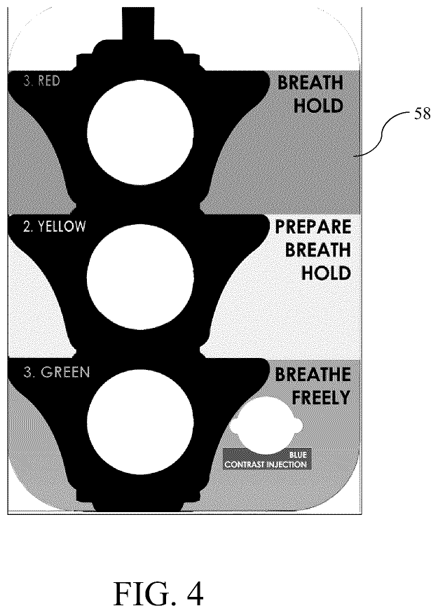

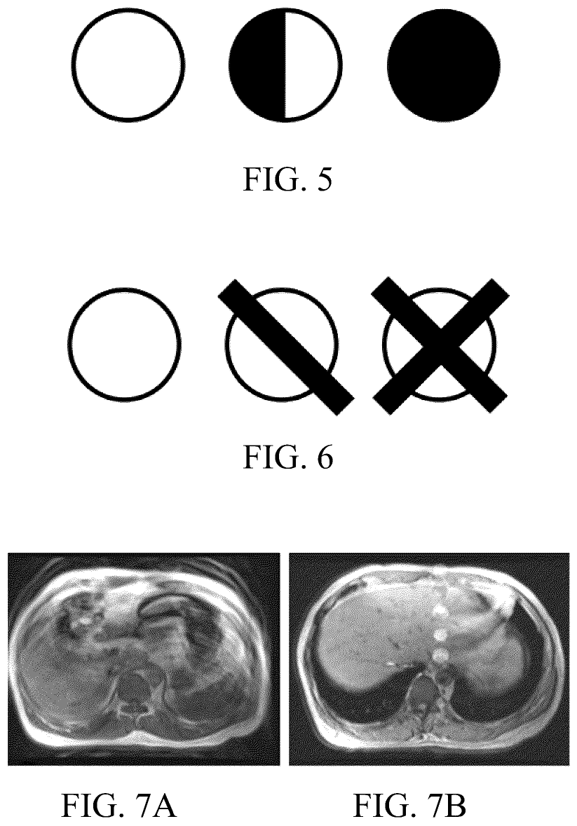

The apparatus 10 can further comprise a display device 50. The display device 50 can be indicative of the message communicated to the individual (patient). For example, in some aspects, and with reference to FIGS. 5-6, the display device comprises at least one symbol that is in communication with the conduit 20 so that the at least one symbol is configured to be illuminated by the at least one light. In further aspects, the display device can comprise a plurality of outlet areas that are configured to be selectively illuminated. The different outlet areas can correspond to different messages (e.g., breathe freely, hold breath, or prepare to hold breath). In exemplary aspects, the different outlet areas can be labeled with text, wherein the message being conveyed to the individual (patient) corresponds to the message adjacent a particular illuminated outlet area. Optionally, the display device can comprise a label 58 (FIG. 4) (e.g., coupled to a housing of the display device 50 via adhesive). Optionally, the label 58 can be a removable label that can be changed to adapt the display device for a different language (e.g., including text instructions in English, Spanish, French, etc.). In some aspects, different outlet areas can be different shapes, for example, in the shape of symbols to convey the message indicated by the illuminated symbol. In some aspects, the different outlet areas can be provided with respective colored windows so that illumination of the different outlet areas have different colors. In some aspects, the different outlet areas can be arranged in a particular order so that the illumination of a particular outlet area in the particular order is indicative of a corresponding message. For example, referring to FIG. 2, the display device can comprise three outlet areas arranged along an axis 57 (like a stoplight). Accordingly, in one example, moving along the axis, the first outlet area can correspond to a message to breathe freely, the second outlet area can indicate a message prepare to hold breath, and the third outlet area can indicate the message to hold one's breath.

Accordingly, in some aspects, the display device 50 can comprise a first outlet area and a second outlet area. A first light can be configured to emit light through the first fiber optic cable to the first outlet area, and the second light can be configured to emit light through the second fiber optic cable to the second outlet area. In further aspects, the display device can comprise a third outlet area, and a third light can be configured to emit light through a third fiber optic cable to the third outlet area.

In still further aspects, the display device 50 can have only a single outlet area. For example, the single illuminated outlet area can correspond to a message to hold one's breath, while the single outlet area not being illuminated can correspond to a message to breathe freely.

In some aspects, the input device 40 can comprise at least one switch 42. For example, the at least one switch can be configured to selectively illuminate each light of the at least one light. In one aspect, the input device 40 can comprise a respective switch for each light of the at least one light. Each switch can be selectively configured to illuminate a corresponding light of the at least one light. It is contemplated that light engines can be associated with input devices, such as remote controllers for selectively activating, deactivating, and controlling color of lights of the light engine.

In some aspects, radio frequency blocking or shielding (e.g., a Faraday Cage) can be provided for light sources used as disclosed herein.

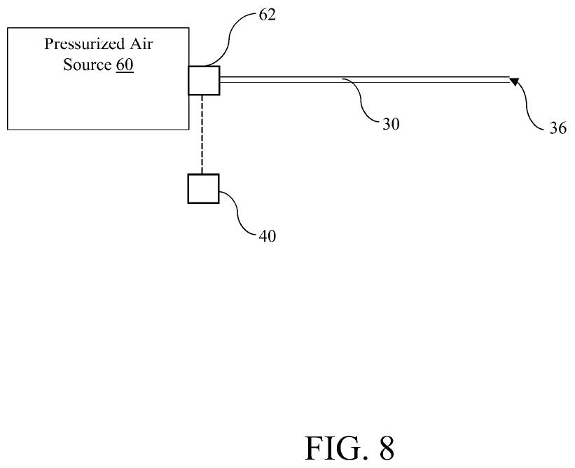

In addition to the disclosed visual feedback, or as an alternative to the disclosed visual feedback, in some exemplary aspects, it is contemplated that the apparatus 10 can provide tactile feedback. In this way, messages can be communicated to visually impaired individuals. For example, the messages can be communicated via pressurized air. In these aspects, and with reference to FIG. 8, the conduit 30 can comprise, or consist of, a tube. The indication source 20 can comprise a pressurized air source 60 and a valve 62 that is configured to selectively release pressurized air from the pressurized air source 60 into the tube. In some aspects, the input device 50 can comprise a switch (e.g., a solenoid switch) that is configured to effect actuation of the valve. In other aspects, the input device 50 can be a handle of a manually actuated valve. The pressurized air source can be medical air or oxygen that is conventionally provided in hospitals. Optionally, the pressurized air can be configured to actuate a pneumatically actuated vibrator. For example, the pneumatically actuated vibrator can be in fluid communication with the outlet of the tube. The pneumatically actuated vibrator can be positioned against patient or near the patient so that the patient can sense vibrations from the pneumatically actuated vibrator.

In aspects in which the message is communicated via pressurized air, the message can relate to a presence or absence of air flowing through the tube. For example, air flowing through the tube can correspond to a message to hold one's breath, and an absence of air flowing through the tube can correspond to a message to breathe freely. In further aspects, air can be pulsed to provide additional information. For example, pulsing of air can indicate that a message to hold one's breath is forthcoming. It is contemplated that an outlet 36 of the conduit 30 (e.g., the tube) can be positioned to deliver pressurized air flow to the head of the patient or to other exposed skin of the patient, or anywhere where the patient can feel airflow from the conduit.

Referring to FIGS. 1-2, the apparatus can further comprise a support structure 70 that is configured to support the second end portion of the conduit relative to the MRI device. The support structure 70 can be free of magnetic metal. Optionally, the apparatus can mount directly to the MRI. In other aspects, the support structure 70 can be a freestanding support structure that rests on the floor. In this way, no modification of the MRI device is required. Accordingly, it is contemplated that the disclosed apparatus, systems, and methods can be used with any MRI scanner design without the need for modification of the structure of the MRI scanner. For example, it is contemplated that the disclosed apparatus, systems, and methods can be used during an MRI procedure performed using a first MRI scanner type, and then subsequently used during an MRI procedure performed using a second MRI scanner type that is different than the first MRI scanner type.

In some aspects, the support structure 70 can comprise an arm 72 that is configured to extend into the MRI device. For example, the display device 50 can couple to the arm of the support structure. In this way, the display device 50 can be inserted into the MRI within view of the individual (patient) in the MRI. The support structure 70 can comprise a flexible support element 74 that permits fine adjustment of the position and angle of the display device 50 relative to the arm 72.

Optionally, the conduit can be at least 5 feet long. In other aspects, the conduit can be at least 10 feet long, at least 15 feet long, at least 20 feet long, at least 25 feet long, or at least 30 feet long. It is contemplated that an entirety of the conduit 30 can be free of magnetic metal (or any material that interferes with an MRI). On other aspects, the conduit can comprise magnetic metal on the first end portion 32. In these aspects, at least the second end portion 34 that is free of magnetic metal can be at least 5 feet long at least 10 feet long, at least 15 feet long, at least 20 feet long, at least 25 feet long, or at least 30 feet long. In this way, any components of the apparatus having magnetic metal can be spaced sufficiently far away from the MRI (optionally, positioned in a different room) to avoid danger during an MRI procedure.

A system 100 can comprise an MRI device 110 and an apparatus 10 as disclosed herein. In some aspects, the second end portion 34 of the conduit 30 can define an outlet 36. The outlet 36 can be positioned above a head of the individual (patient). For example, the outlet 36 can be positioned within the display device 50, with the display device positioned above the head of the individual (patient).

A method of communicating with an individual (patient) in an MRI includes the step of delivering, to the individual (patient) in an MRI device, via a conduit having a second end portion is free of magnetic metal and extends to an MRI device, a message in the form of one of pressurized air or light. In some aspects, the message is associated with an indication for an individual (patient) in the MRI device to commence breath holding.

In some aspects, the message comprises light. In these aspects, delivering the message to the individual (patient) comprises illuminating at least one light to generate the light and conducting the light form the at least one light to the MRI device via a fiber optic cable. In other aspects, the message comprises pressurized air. In these aspects, delivering the message to the individual (patient) comprises releasing pressurized air from a pressurized air source.

Optionally, the method comprises, prior to beginning an MRI procedure, explaining a requested action of the individual (patient) in response to receiving the message. For example, while a patient has hearing aids, a medical professional can explain what the light or pressurized air messages correspond to. Then, the patient can remove his/her hearing aids and enter the MRI.

Hearing loss and impairment contribute to the present problem, primarily affecting thoracic, lumbar and pelvis imaging. FIGS. 7A-7B illustrate advantages of using the disclosed apparatuses, system, and method. FIG. 7A is an exemplary poor quality image of a patient who was breathing during imaging. FIG. 7B is an exemplary high quality image of a patient who was properly holding her breath during imaging. As can be seen by comparing FIGS. 7A and 7B, 7A shows blurred regions associated with different portions of the patient's body being captured at different times, the different portions moving due to the patient's breathing.

### EXEMPLARY ASPECTS

In view of the described products, systems, and methods and variations thereof, herein below are described certain more particularly described aspects of the invention. These particularly recited aspects should not however be interpreted to have any limiting effect on any different claims containing different or more general teachings described herein, or that the “particular” aspects are somehow limited in some way other than the inherent meanings of the language literally used therein.

Aspect 1: An apparatus for communicating a message to a patient in a magnetic resonance imaging (MRI) device, the apparatus comprising:
an indication source;
a conduit having a first end portion and a second end portion, wherein the first end portion is in communication with the indication source, wherein at least the second end portion is free of magnetic metal; and
an input device configured to receive an input from a medical professional, wherein the input device is in operable communication with the indication source so that receipt of the input from the medical professional causes the indication source to output a communicating signal through the conduit.

Aspect 2: The apparatus of aspect 1, wherein the indication source comprises at least one light, wherein the conduit is a fiber optic cable.

Aspect 3: The apparatus of aspect 2, wherein the at least one light comprises a color-changing light.

Aspect 4: The apparatus of aspect 2, wherein the at least one light comprises a first light and a second light, wherein the fiber optic cable is a first fiber optic cable, wherein the apparatus further comprises a second fiber optic cable, wherein the first light is configured to emit light through the first fiber optic cable, wherein the second light is configured to emit light through the second fiber optic cable.

Aspect 5: The apparatus of any one of aspects 2-4, further comprising a display device, wherein the display device is indicative of the message communicated to the individual.

Aspect 6: The apparatus of aspect 5, wherein the display device comprises at least one symbol that is configured to be illuminated by the at least one light.

Aspect 7: The apparatus of aspect 5, wherein the display device comprises a first outlet area and a second outlet area, wherein the at least one light comprises a first light and a second light, wherein the fiber optic cable is a first fiber optic cable, wherein the apparatus further comprises a second fiber optic cable, wherein the first light is configured to emit light through the first fiber optic cable to the first outlet area, wherein the second light is configured to emit light through the second fiber optic cable to the second outlet area.

Aspect 8: The apparatus of aspect 7, wherein the display device further comprises comprising a third outlet area, wherein the first, second, and third outlet areas are arranged along a first axis.

Aspect 9: The apparatus of aspect 7, wherein the first and second outlet areas each have respective windows, wherein the respective windows of the first and second outlet areas are different colors.

Aspect 10: The apparatus of any one of the preceding aspects, wherein the input device comprises at least one switch.

Aspect 11: The apparatus of any one of aspects 2-10, wherein the input device comprises a respective switch for each light of the at least one light, wherein each switch is selectively configured to illuminate a corresponding light of the at least one light.

Aspect 12: The apparatus of aspect 1, wherein the conduit is a tube, wherein the indication source comprises a pressurized air source and a valve that is configured to selectively release pressurized air into the conduit.

Aspect 13: The apparatus of aspect 12, wherein the input device comprises a switch that is configured to effect actuation of the valve.

Aspect 14: The apparatus of any one of aspects 1-13, further comprising a support structure that is configured to support the second end portion of the conduit relative to the MRI device, wherein the support structure is free of magnetic metal.

Aspect 15: The apparatus of aspect 14, wherein the support structure comprises an arm that is configured to extend into the MRI device.

Aspect 16: The apparatus of aspect 15, further comprising a display device, wherein the display device is coupled to the arm of the support structure.

Aspect 17: The apparatus of any one of aspects 1-16, wherein the conduit is at least 15 feet long.

Aspect 18: A system comprising:
an MRI device;
an apparatus as in any one of the preceding aspects.

Aspect 19: The system of aspect 18, wherein the second end portion of the conduit defines an outlet, wherein the outlet is positioned above a head of the individual.

Aspect 20: A method comprising:
delivering, to a patient in an MRI device, via a conduit having a second end portion is free of magnetic metal and extends to an MRI device, a message in the form of one of pressurized air or light.

Aspect 21: The method of aspect 20, wherein the message comprises light, wherein delivering the message to the patient comprises:
illuminating at least one light to generate the light; and
conducting the light form the at least one light to the MRI device via a fiber optic cable.

Aspect 22: The method of aspect 20, wherein the message comprises pressurized air, wherein delivering the message to the patient comprises releasing pressurized air from a pressurized air source.

Aspect 23: The method of aspect 20, wherein the message is associated with an indication for a patient in the MRI device to commence breath holding.

Those skilled in the art will recognize, or be able to ascertain using no more than routine experimentation, many equivalents to the specific embodiments of the method and compositions described herein. Such equivalents are intended to be encompassed by the following claims.

What is claimed is:

1. An apparatus for communicating a message to a patient in a magnetic resonance imaging (MRI) device, the apparatus comprising:
an indication source;
a conduit having a first end portion and a second end portion, wherein the first end portion is in communication with the indication source, wherein at least the second end portion is free of magnetic metal; and
an input device configured to receive an input from a medical professional, wherein the input device is in operable communication with the indication source so that receipt of the input from the medical professional causes the indication source to output a communicating signal through the conduit.

2. The apparatus of claim 1, wherein the indication source comprises at least one light, wherein the conduit is a fiber optic cable.

3. The apparatus of claim 2, wherein the at least one light comprises a color-changing light.

4. The apparatus of claim 2, wherein the at least one light comprises a first light and a second light, wherein the fiber optic cable is a first fiber optic cable, wherein the apparatus further comprises a second fiber optic cable, wherein the first light is configured to emit light through the first fiber optic cable, wherein the second light is configured to emit light through the second fiber optic cable.

5. The apparatus of claim 2, further comprising a display device, wherein the display device is indicative of the message communicated to the individual.

6. The apparatus of claim 5, wherein the display device comprises at least one symbol that is configured to be illuminated by the at least one light.

7. The apparatus of claim 5, wherein the display device comprises a first outlet area and a second outlet area, wherein the at least one light comprises a first light and a second light, wherein the fiber optic cable is a first fiber optic cable, wherein the apparatus further comprises a second fiber optic cable, wherein the first light is configured to emit light through the first fiber optic cable to the first outlet area, wherein the second light is configured to emit light through the second fiber optic cable to the second outlet area.

8. The apparatus of claim 7, wherein the display device further comprises comprising a third outlet area, wherein the first, second, and third outlet areas are arranged along a first axis.

9. The apparatus of claim 7, wherein the first and second outlet areas each have respective windows, wherein the respective windows of the first and second outlet areas are different colors.

10. The apparatus of claim 1, wherein the input device comprises at least one switch.

11. The apparatus of claim 2, wherein the input device comprises a respective switch for each light of the at least one light, wherein each switch is selectively configured to illuminate a corresponding light of the at least one light.

12. The apparatus of claim 1, wherein the conduit is a tube, wherein the indication source comprises a pressurized air source and a valve that is configured to selectively release pressurized air into the conduit.

13. The apparatus of claim 12, wherein the input device comprises a switch that is configured to effect actuation of the valve.

14. The apparatus of claim 1, further comprising a support structure that is configured to support the second end portion of the conduit relative to the MRI device, wherein the support structure is free of magnetic metal.

15. The apparatus of claim 14, wherein the support structure comprises an arm that is configured to extend into the MRI device.

16. The apparatus of claim 15, further comprising a display device, wherein the display device is coupled to the arm of the support structure.

17. The apparatus of claim 1, wherein the conduit is at least 15 feet long.

18. A system comprising:
an MRI device;
an apparatus comprising:
an indication source;
a conduit having a first end portion and a second end portion, wherein the first end portion is in communication with the indication source, wherein at least the second end portion is free of magnetic metal; and
an input device configured to receive an input from a medical professional, wherein the input device is in operable communication with the indication source so that receipt of the input from the medical professional causes the indication source to output a communicating signal through the conduit.

19. A method comprising:
delivering, to a patient in an MRI device, via a conduit having a second end portion is free of magnetic metal and extends to an MRI device, a message in the form of one of pressurized air or light.

20. The method of claim 19, wherein the message is associated with an indication for a patient in the MRI device to commence breath holding.
